(19) United States
(12) Patent Application Publication
(10) Pub. No.: US 2002/0002298 A1
(43) Pub. Date: Jan. 3, 2002

(54) Process for the production of esterquats

(76) Inventors: Joaquim Bigorra Llosas, Sabadell (ES); Rafael Pi Subirana, Granollers (ES); Nuria Bonastre Gilabert, Barbera del Valles (ES)

Correspondence Address: COGNIS CORPORATION, 2500 RENAISSANCE BLVD., SUITE 200, GULPH MILLS, PA, 19406

(21) Appl. No.: 09/854,106

(22) Filed: May 11, 2001

(30) Foreign Application Priority Data
May 11, 2000 (DE) DE100 22 966.2

(51) Int. Cl. C07C 229/06; C11D 1/38

(52) U.S. Cl. 554/114

(57) ABSTRACT

The invention relates to a process for the production of esterquats in which triglycerides are transesterified with alkanolamines and the resulting fatty acid alkanolamine esters are subsequently quaternized with alkyl halides or dialkyl sulfates in the presence of solvents, characterized in that glycerol released during the transesterification is continuously removed from the reaction equilibrium.

### FIELD OF THE INVENTION

This invention relates generally to cationic surfactants and more particularly to an improved process for the production of esterquats starting from triglycerides.

### PRIOR ART

“Esterquats” are generally understood to be quaternized fatty acid alkanolamine ester salts which, in recent years, have proved to be suitable as another group of cationic surfactants—besides the known tetraalkyl ammonium salts—for use as fabric softeners and as conditioners for cosmetics. They are normally produced by condensation of fatty acids with alkanolamines and subsequent quaternization of the esters with methyl chloride or dimethyl sulfate. Reference is made in this connection to International patent application WO 91/01295 (Henkel), according to which triethanolamine is partly esterified with fatty acids in the presence of hypophosphorous acid, air is passed through and the reaction product is quaternized with dimethyl sulfate or ethylene oxide. In addition, German patent DE 4308794 C1 (Henkel) describes a process for the production of solid esterquats in which the quaternization of triethanolamine esters is carried out in the presence of suitable dispersants, preferably fatty alcohols. Overviews on this subject have been published, for example, by R. Puchta et al. in Tens. Surf. Det., 30, 186 (1993), by M. Brock in Tens. Surf. Det. 30, 394 (1993), by R. Lagerman et al. in J. Am. Oil. Chem. Soc., 71, 97 (1994) and by I. Shapiro in Cosm. Toil, 109, 77 (1994).

A particularly effective process for the production of esterquats is proposed in European patent EP 0 750 606 B1 (Cognis). In this process, the fatty acids are replaced by triglycerides which are transesterified with the alkanolamines to the fatty acid alkanolamine esters and then quaternized. The advantage of this process lies above all in the fact that it uses relatively unrefined starting materials. Unfortunately, its disadvantage is that the resulting esterquats have poorer performance properties than products of comparable composition which have been obtained by esterification. The outcome of this has been that, despite lower production costs, the transesterification products have not hitherto been successfully used.

Accordingly, the problem addressed by the present invention was to improve the process according to EP 0 750 606 B1 in such a way that products capable of competing in their performance properties would be obtained without losing the advantage in relation to production costs.

### DESCRIPTION OF THE INVENTION

The present invention relates to a process for the production of esterquats in which triglycerides are transesterified with alkanolamines and the resulting fatty acid alkanolamine esters are subsequently quaternized with alkyl halides or dialkyl sulfates in the presence of solvents, characterized in that glycerol released during the transesterification is continuously removed from the reaction equilibrium.

In extensive studies, applicants have found that the disadvantages in the performance properties of the transesterification products are attributable to the presence of partial glycerides. By the simple measure of continuously removing the glycerol released during the transesterification from the reaction mixture and thus eliminating the source of partial glycerides, esterquats no longer inferior to the esterification products in their properties are now also obtained on the basis of triglycerides. The additional expenditure on the distillation step does not add significantly to the production costs, especially since the glycerol removed may be completely or partly used as a solvent for the esters in the quaternization.

Transesterification

Basically, the esterquats can be produced from any synthetic triglycerides or natural fats and oils which correspond to general formula (I):

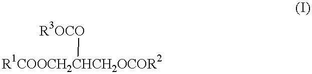

in which R¹CO, R²CO and R³CO independently of one another represent linear and/or branched, saturated and/or unsaturated acyl groups containing 6 to 22 and preferably 12 to 18 carbon atoms and 0 and/or 1 to 3 double bonds. The triglycerides are preferably hydrogenated or at least partly hydrogenated, i.e. have iodine values of 0 or 0.5 to 40 and more particularly 5 to 25. Suitable starting materials are, for example, palm kernel oil, olive oil and the like, the use of bovine tallow, coconut oil and palm oil, optionally after a hardening step, being preferred. Suitable alkanolamines are, above all, triethanolamine (TEA) or methyl diethanolamine (MDA) and mixtures thereof. The transesterification is carried out in known manner using mainly alkaline catalysts such as, for example, alkali metal hydroxides, alkali metal alcoholates, zinc soaps and the like. To produce the quaternized esters, the fatty acids and the triethanolamine may be used in a molar ratio of 1.1:1 to 3:1. With the performance properties of the esterquats in mind, a ratio of 1.2:1 to 2.2:1 and preferably 1.5:1 to 1.9:1 has proved to be particularly advantageous. The preferred esterquats are technical mixtures of mono-, di- and triesters with an average degree of esterification of 1.5 to 1.9.

Quaternization

The quaternization may also be carried out in known manner. It has proved to be of advantage to introduce the alkanolamine esters into a suitable solvent beforehand. Preferred solvents are lower aliphatic alcohols such as, for example, isopropyl alcohol or ethylene glycol which are not problematical from the performance perspective and are also safe where the products are used for cosmetic applications. In one preferred embodiment of the invention, the glycerol released during the transesterification and subsequently removed is returned to the quaternization stage as solvent for the esters. In this way, the additional cost of the distillation step can mostly be recovered. Suitable quaternizing agents are alkali metal halides, such as methyl chloride for example, or dialkyl sulfates, such as dimethyl sulfate for example. Further particulars of how the transesterification and quaternization steps are carried out can be found in the above-cited EP 0 750 606 B1 to the entire disclosure of which reference is hereby specifically made.

### EXAMPLES

### Example 1

780 g (0.92 mol) of bovine tallow, 175 g (1.17 mol) of triethanolamine and 1.4 g of potassium hydroxide in the form of a 45% by weight aqueous solution were introduced into a 2-liter distillation assembly and heated to 80° C. Vacuum was then applied and the pressure reduced to 2 mm Hg while the temperature was continuously increased to 180° C. Another 75 g (0.5 mol) of triethanolamine were then added. 81.5 g (=96% of the theoretical) of glycerol were distilled off over a period of 2 hours. The vacuum was then broken, the mixture was cooled and 200 g (0.34 mol) of the tallow fatty acid triethanolamine ester formed were transferred to a stirred reactor. Here, the ester was dissolved in a mixture of 18 g of glycerol and 46 g of isopropyl alcohol and 41 g (0.32 mol) of dimethyl sulfate were added to the resulting solution in portions with stirring over a period of 4 hours at 65° C. The esterquat preparation had a dry residue of 85% by weight.

### Example 2

A tallow fatty acid triethanolamine ester was prepared as described in Example 1 and dissolved in 63 g of isopropyl alcohol. The resulting esterquat preparation had a dry residue of 90% by weight.

1. A process for the production of esterquats in which triglycerides are transesterified with alkanolamines and the resulting fatty acid alkanolamine esters are subsequently quaternized with alkyl halides or dialkyl sulfates in the presence of solvents, characterized in that glycerol released during the transesterification is continuously removed from the reaction equilibrium.

2. A process as claimed in claim 1, characterized in that triglycerides corresponding to formula (I): 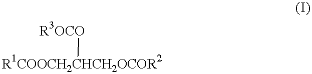
in which R¹CO, R²CO and R³CO independently of one another represent linear and/or branched, saturated and/or unsaturated acyl groups containing 6 to 22 and preferably 12 to 18 carbon atoms and 0 and/or 1 to 3 double bonds, are used.

3. A process as claimed in claims 1 and/or 2, characterized in that bovine tallow, coconut oil or palm oil is used as the triglyceride.

4. A process as claimed in at least one of claims 1 to 3, characterized in that triethanolamine (TEA) and/or methyl diethanolamine (MDA) is/are used as the alkanolamines.

5. A process as claimed in at least one of claims 1 to 3, characterized in that the triglycerides and the alkanolamines are used in a molar ratio of 1.1:1 to 3:1, based on the fatty acid groups.

6. A process as claimed in at least one of claims 1 to 4, characterized in that the glycerol removed during the transesterification is completely or partly used as solvent in the quaternization step.
